(19) United States
(12) Patent Application Publication
(10) Pub. No.: US 2024/0387060 A1
(43) Pub. Date: Nov. 21, 2024

(54) Drug Sensitivity Prediction and Model Training Method, Storage Medium and Device

(71) Applicant: BOE Technology Group Co., Ltd., Beijing (CN)

(72) Inventors: Pi CAO, Beijing (CN); Shuobin LIANG, Beijing (CN)

(21) Appl. No.: 18/034,071

(22) PCT Filed: May 20, 2022
(86) PCT No.: PCT/CN2022/094234

§ 371 Date: Apr. 27, 2023

(51) Int. Cl. G16H 70/40; G16B 20/50

(52) U.S. Cl. CPC G16H 70/40; G16B 20/50

(57) ABSTRACT

A method for predicting drug sensitivity includes: acquiring gene expression information of a cell line to be tested, gene mutation information of the cell line to be tested, and structural information of a drug to be tested; calculating first correlation information between the structural information of the drug to be tested and the gene expression information based on a first attention model; calculating second correlation information between the structural information of the drug to be tested and the gene mutation information based on a second attention model; splicing the first correlation information and the second correlation information to obtain a splicing result; and performing a prediction processing on the splicing result based on a drug sensitivity prediction model to obtain sensitivity information of the cell line to be tested for the drug to be tested.

### CROSS-REFERENCE TO RELATED APPLICATION

This application is a national stage application of PCT Application No. PCT/CN2022/094234, which is filed on May 20, 2022, and entitled “Drug Sensitivity Prediction and Model Training Method, Storage Medium and Device”, the content of which should be regarded as being incorporated herein by reference.

### TECHNICAL FIELD

Embodiments of the present disclosure relate to, but are not limited to, the technical field of big data processing, in particular to a drug sensitivity prediction and model training method, a storage medium and a device.

### BACKGROUND

For drugs used to treat tumors, a low sensitivity of the drugs is one of the important reasons for their treatment failure, and a decrease in sensitivity of the drugs is also one of influential factors in tumor recurrence. At present, concomitant diagnostic products of anti-tumor drugs focus on targeting drugs, and the main mechanism thereof is to detect a type of gene mutation in patients and recommend drugs according to the results of mutation. Therefore, drug sensitivity analysis based on large-scale pharmacogenomic data is one of the current research directions. The Genomics of Drug Sensitivity in Cancer (GDSC) database and the Broad Institute Cancer Cell Line Encyclopedia (CCLE) database, which contain mutation information, expression information, copy number variation, methylation information and drug dose response data of tumor cell lines, have become one of the most important tools. With a rise of the method of deep learning and further verification of its ability to learn the richest information from original data, it is imperative to predict drug sensitivity using deep learning.

### SUMMARY

The following is a summary of subject matters described herein in detail. The summary is not intended to limit the protection scope of claims.

In a first aspect, an embodiment of the present disclosure provides a method for predicting drug sensitivity, including:
acquiring gene expression information of a cell line to be tested, gene mutation information of the cell line to be tested, and structural information of a drug to be tested;
calculating first correlation information between the structural information of the drug to be tested and the gene expression information based on a first attention model; and calculating second correlation information between the structural information of the drug to be tested and the gene mutation information based on a second attention model;
splicing the first correlation information and the second correlation information to obtain a splicing result; and
performing a prediction processing on the splicing result based on a drug sensitivity prediction model to obtain sensitivity information of the cell line to be tested for the drug to be tested.

In a second aspect, an embodiment of the present disclosure further provides a method for training a drug sensitivity prediction model, including:
acquiring a training sample set, the training sample set including gene expression information of a cell line, gene mutation information of the cell line, structural information of a plurality of drugs and a plurality of pieces of reference semi-inhibitory concentration information, wherein each piece of reference semi-inhibitory concentration information corresponds to the structural information of one drug;
obtaining a plurality of pieces of first prediction related information between the structural information of the plurality of drugs and the gene expression information respectively based on a first attention model; and obtaining a plurality of pieces of second prediction related information between the structural information of the plurality of drugs and the gene mutation information respectively based on a second attention model;
splicing the first prediction related information and the second prediction related information corresponding to the structural information of a same drug to obtain a plurality of spliced prediction results; and
training a prediction model to be trained by using the plurality of spliced prediction results and the plurality of pieces of reference semi-inhibitory concentration information to obtain a drug sensitivity prediction model.

In an exemplary implementation, the acquiring a training sample set includes:
acquiring raw data of the gene expression information, the raw data of the gene expression information including average values of a plurality of first gene expression features and standard deviations of the plurality of first gene expression features;
normalizing the average values of the plurality of first gene expression features to obtain a plurality of normalized expression average values, normalizing the standard deviations of the plurality of first gene expression features to obtain a plurality of normalized expression standard deviations, and inputting the plurality of normalized standard deviations and the plurality of normalized expression average values into an encoder;
controlling the encoder to add or subtract the normalized expression standard deviations corresponding to the normalized expression average values to or from a part of the normalized expression average values to obtain a plurality of processed normalized expression average values, and taking another part of unprocessed normalized expression average values and the plurality of processed normalized expression average values as a plurality of encoding input features; and
controlling the encoder to encode the plurality of encoding input features to obtain a plurality of second gene expression features as the gene expression information, the number of the plurality of second gene expression features being less than the number of the plurality of first gene expression features.

In an exemplary implementation, the encoder includes an encoding layer, and the encoding layer includes an input layer and an output layer;
the controlling the encoder to encode the plurality of encoding input features includes:
controlling the encoder to perform the following operation on the plurality of encoding input features to obtain a plurality of second gene expression features: y=s (Wx̅+b), wherein x̅ is the encoding input feature, y is the second gene expression feature, W is a link weight from the input layer to the output layer, b is a deviation of the output layer, and s is a nonlinear function.

In an exemplary implementation, prior to the inputting the plurality of normalized standard deviations and the plurality of normalized expression average values into the encoder, the method further includes:
acquiring a training sample set of the first gene expression features, the training sample set of the first gene expression features including average value samples of a plurality of first gene expression features and corresponding standard deviation samples of the plurality of first gene expression features, and training the encoder according to the average value samples of the plurality of first gene expression features and the standard deviation samples of the plurality of first gene expression features to obtain a link weight W, a deviation b of the output layer and a nonlinear function s.

In an exemplary implementation, the training the encoder according to the average value samples of the plurality of first gene expression features and the standard deviation samples of the plurality of first gene expression features includes:
inputting a plurality of spliced prediction results into an encoder to be trained for a plurality of times through multiple iterations, and optimizing the encoder to be trained according to a result of each iteration.

In an exemplary implementation, the encoder further includes a decoding layer;
the inputting a plurality of spliced prediction results into an encoder to be trained for a plurality of times through multiple iterations, and optimizing the encoder to be trained according to a result of each iteration includes:
normalizing the average value samples of the plurality of first gene expression features to obtain normalized expression average value samples, normalizing the standard deviation samples of the plurality of first gene expression features to obtain normalized expression standard deviation samples, and inputting the plurality of normalized standard deviation samples and the plurality of normalized expression average value samples into the encoder to be trained;
controlling the encoder to be trained to add or subtract the expression standard deviation samples corresponding to the normalized expression average value samples to or from a part of the normalized expression average value samples to obtain a plurality of processed normalized expression average value samples, and taking another part of unprocessed normalized expression average value samples and the plurality of processed normalized expression average value samples as a plurality of encoding input feature samples;
controlling the encoder to be trained to encode the plurality of encoding input feature samples to obtain a plurality of second gene expression feature samples as gene expression information samples, the number of the plurality of second gene expression feature samples being less than the number of the plurality of first gene expression feature samples;
inputting the gene expression information samples into the decoding layer to obtain decoding information; and
calculating a loss value according to the decoding information and the average value samples of the plurality of first gene expression features, optimizing the encoder according to the loss value, using the optimized encoder as an encoder to be trained in a next iteration, and continuously controlling the encoder to be trained to add or subtract the expression standard deviation samples corresponding to the normalized expression average value samples to or from a part of the normalized expression average value samples.

In an exemplary implementation, the inputting the gene expression information into the decoding layer to obtain decoding information includes:
controlling the encoder to be trained to perform the following operation on the gene expression information to obtain the decoding information: z=s (W′y+b′), wherein s is a nonlinear function, W′ is a link weight of the decoding layer, b′ is a deviation of the decoding layer, y is a feature value in the gene expression information, and z is a feature value of the decoding information;
the calculating a loss value according to the decoding information and the average value samples of the plurality of first gene expression features includes:
controlling the encoder to be trained to perform the following operation according to the average value samples of the plurality of first gene expression features and the decoding information to obtain the loss value: L(x,z)=x−z∥², wherein L(x,z) is a loss function, x is a feature value in the average value samples of the first gene expression features, and z is a feature value of the decoding information.

In a third aspect, an embodiment of the present disclosure further provides a non-transitory computer-readable storage medium, the storage medium being configured to store computer program instructions, wherein when the computer program instructions are run, the method for predicting drug sensitivity according to any one of the aforementioned embodiments is implemented, or when the computer program instructions are run, the method for training a drug sensitivity prediction model according to any one of the aforementioned embodiments is implemented.

In a fourth aspect, an embodiment of the present disclosure further provides a device for predicting drug sensitivity, including a first memory, a first processor, and a computer program stored on the first memory and runnable on the first processor, to perform:
acquiring gene expression information of a cell line to be tested, gene mutation information of the cell line to be tested, and structural information of a drug to be tested;
calculating first correlation information between the structural information of the drug to be tested and the gene expression information based on a first attention model; and calculating second correlation information between the structural information of the drug to be tested and the gene mutation information based on a second attention model;
splicing the first correlation information and the second correlation information to obtain a splicing result; and
performing a prediction processing on the splicing result based on a drug sensitivity prediction model to obtain sensitivity information of the cell line to be tested for the drug to be tested.

In a fifth aspect, an embodiment of the present disclosure further provides a device for training a drug sensitivity prediction model, including a second memory, a second processor, and a computer program stored on the second memory and runnable on the second processor, to perform:
acquiring a training sample set, the training sample set including gene expression information of a cell line, gene mutation information of the cell line, structural information of a plurality of drugs and a plurality of pieces of reference semi-inhibitory concentration information, wherein each piece of reference semi-inhibitory concentration information corresponds to the structural information of one drug;
obtaining a plurality of pieces of first prediction related information between the structural information of the plurality of drugs and the gene expression information respectively based on a first attention model; and obtaining a plurality of pieces of second prediction related information between the structural information of the plurality of drugs and the gene mutation information respectively based on a second attention model;
splicing the first prediction related information and the second prediction related information corresponding to the structural information of a same drug to obtain a plurality of spliced prediction results; and
training a prediction model to be trained by using the plurality of spliced prediction results and the plurality of pieces of reference semi-inhibitory concentration information to obtain a drug sensitivity prediction model.

In a sixth aspect, an embodiment of the present disclosure further provides an apparatus for predicting drug sensitivity, including:
an acquiring module, configured to acquire gene expression information of a cell line to be tested, gene mutation information of the cell line to be tested, and structural information of a drug to be tested;
a first feature fusion module, configured to calculate first correlation information between the structural information of the drug to be tested and the gene expression information based on a first attention model;
a second feature fusion module, configured to calculate second correlation information between the structural information of the drug to be tested and the gene mutation information based on a second attention model;
a splicing module, configured to splice the first correlation information and the second correlation information to obtain a splicing result; and
a drug sensitivity prediction module, configured to perform a prediction processing on the splicing result based on a drug sensitivity prediction model to obtain sensitivity information of the cell line to be tested for the drug to be tested.

In a seventh aspect, an embodiment of the present disclosure further provides an apparatus for training a drug sensitivity prediction model, including:
a sample acquisition module, configured to acquire a training sample set, the training sample set including gene expression information of a cell line, gene mutation information of the cell line, structural information of a plurality of drugs and a plurality of pieces of reference semi-inhibitory concentration information, wherein each piece of reference semi-inhibitory concentration information corresponds to the structural information of one drug;
a first feature fusion prediction module, configured to obtain a plurality of pieces of first prediction related information between the structural information of the plurality of drugs and the gene expression information respectively based on a first attention model;
a second feature fusion prediction module, configured to obtain a plurality of pieces of second prediction related information between the structural information of the plurality of drugs and the gene mutation information respectively based on a second attention model;
a splicing prediction module, configured to splice the first prediction related information and the second prediction related information corresponding to the structural information of a same drug to obtain a plurality of spliced prediction results; and
a sensitivity prediction module, configured to train a prediction model to be trained by using the plurality of spliced prediction results and the plurality of pieces of reference semi-inhibitory concentration information to obtain a drug sensitivity prediction model.

Other aspects may be understood upon reading and understanding the drawings and detailed description.

### BRIEF DESCRIPTION OF DRAWINGS

The drawings are intended to provide a further understanding of technical solutions of the present disclosure and form a part of the specification, and are used to explain the technical solutions of the present disclosure together with embodiments of the present disclosure, and do not form limitations on the technical solutions of the present disclosure. Shape and size of each component in the drawings do not reflect actual scales, and are only intended to schematically illustrate contents of the present disclosure.

FIG. 1 is a flowchart of a method for predicting drug sensitivity according to an embodiment of the present disclosure.

FIG. 2 is a flowchart of a method for predicting drug sensitivity according to an exemplary embodiment of the present disclosure.

FIG. 3 is a schematic diagram of structure of an encoding layer of an encoder according to an exemplary embodiment of the present disclosure.

FIG. 4 is a flowchart of a method for training a drug sensitivity prediction model according to an embodiment of the present disclosure.

FIG. 5 is a schematic diagram of structure of an encoder according to an exemplary embodiment of the present disclosure.

FIG. 6 is a schematic diagram of structure of an apparatus for predicting drug sensitivity according to an embodiment of the present disclosure.

FIG. 7 is a schematic diagram of structure of an apparatus for training a drug sensitivity prediction model according to an embodiment of the present disclosure.

FIG. 8 is a schematic diagram of structure of a device for predicting drug sensitivity according to an embodiment of the present disclosure.

FIG. 9 is a schematic diagram of structure of a device for training a drug sensitivity prediction model according to an embodiment of the present disclosure.

FIG. 10 is a schematic diagram of logical structure of drug sensitivity prediction according to an exemplary embodiment of the present disclosure.

### DETAILED DESCRIPTION

The embodiments of the present disclosure will be described in detail below with reference to the drawings. Implementations may be carried out in a plurality of different forms. Those of ordinary skills in the art may easily understand such a fact that implementations and contents may be transformed into various forms without departing from the purpose and scope of the present disclosure. Therefore, the present disclosure should not be explained as being limited to contents described in following implementations only. The embodiments in the present disclosure and features in the embodiments may be combined randomly with each other without conflict. In order to keep following description of the embodiments of the present disclosure clear and concise, detailed descriptions about part of known functions and known components are omitted in the present disclosure. The drawings of the embodiments of the present disclosure only involve structures involved in the embodiments of the present disclosure, and for other structures, reference may be made to conventional designs.

Ordinal numerals such as “first”, “second”, and “third” in the specification are set to avoid confusion of constituent elements, but not to set a limit in quantity.

In the specification, “electrical connection” includes a case that constituent elements are connected together through an element with a certain electrical effect. The “element with a certain electrical effect” is not particularly limited as long as electrical signals may be sent and received between the connected constituent elements. Examples of the “element with a certain electrical effect” not only include an electrode and a wiring, but also may include a switch element such as a transistor, a resistor, an inductor, a capacitor, another element having one or more functions, and the like.

Data used for predicting sensitivity of anti-tumor drugs based on deep learning includes Messenger RibonucleicAcid (mRNA) expression information, mutation information, chemical structural information of the drugs, copy number variation information, etc. Among them, the mRNA expression information is subjected to feature extraction and compression through an Autoencoder, spliced with other information (simple network processing may be involved) based on this, and then input into a final fully connected network for prediction. This prediction mode is prone to feature sparseness, and loses information between data. Moreover, only part information of the mRNA expression information is used when using Autoencoders for feature extraction and compression, which has a defect of poor performance in drug sensitivity prediction.

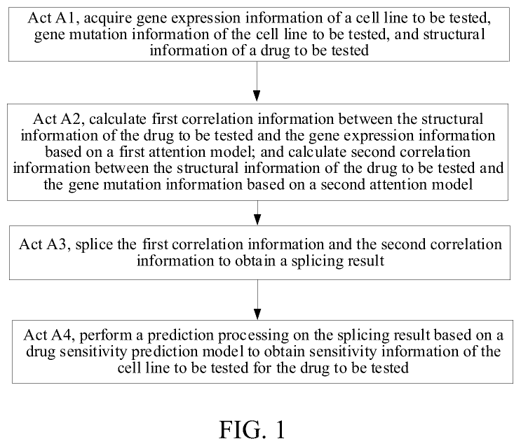

An embodiment of the present disclosure provides a method for predicting drug sensitivity. As shown in FIG. 1, the method for predicting drug sensitivity provided by an embodiment of the present disclosure may include:
act A1, acquiring gene expression information of a cell line to be tested, gene mutation information of the cell line to be tested, and structural information of a drug to be tested;
act A2, calculating first correlation information between the structural information of the drug to be tested and the gene expression information based on a first attention model; and
calculating second correlation information between the structural information of the drug to be tested and the gene mutation information based on a second attention model;
act A3, splicing the first correlation information and the second correlation information to obtain a splicing result; and
act A4, performing a prediction processing on the splicing result based on a drug sensitivity prediction model to obtain sensitivity information of the cell line to be tested for the drug to be tested.

In the method for predicting drug sensitivity provided by an embodiment of the present disclosure, first correlation information between structural information of a drug to be tested and gene expression information is obtained through a first attention model, second correlation information between the structural information of the drug to be tested and gene mutation information is obtained through a second attention model, the first correlation information and the second correlation information are spliced to obtain a splicing result, and the splicing result is processed through a drug sensitivity prediction model to obtain sensitivity information of a cell line to be tested for the drug to be tested. Prior to the prediction through the drug sensitivity prediction model, correlation information between the gene expression information, the gene mutation information and the structural information of the drug is obtained through an attention mechanism, and prediction of drug sensitivity is performed according to the correlation information, which may improve prediction effect of the drug sensitivity prediction model, overcoming the defect of poor effect in drug sensitivity prediction.

In an exemplary implementation, in act A2, the calculating first correlation information between the structural information of the drug to be tested and the gene expression information based on a first attention model may include:
act A201, multiplying the gene expression information by a first weight matrix to obtain a first vector, multiplying the drug structural information by a second weight matrix to obtain a second vector, and multiplying the structural information of the drug by a third weight matrix to obtain a third vector; and
act A202, normalizing the first vector and the second vector to obtain a first processing result, and multiplying the first processing result by the third vector to obtain the first correlation information.

In an exemplary implementation, in act A202, the normalizing the first vector and the second vector to obtain a first processing result includes: transposing the second vector to obtain a transposed vector of the second vector, multiplying the first vector by the transposed vector of the second vector to obtain a first product, and dividing the first product by a first constant to obtain a first processing result, the first constant being an arithmetic square root of a dimensionality of the second vector.

For example, in act A201, Q is set as the gene expression information, K and V are set as the structural information of the drug, the first weight matrix is set as W^{Q}, the second weight matrix is set as W^{K}, and the third weight matrix is set as W^{V}, then the gene expression information Q is multiplied by the first weight matrix W^{Q} to obtain the first vector q=Q*W^{Q}, the structural information K of the drug is multiplied by the second weight matrix W^{K} to obtain the second vector k=K*W^{K}, and the structural information V of the drug is multiplied by the third weight matrix W^{V} to obtain the third vector v=V*W^{V}. The first vector q may be understood as a query vector of a first self-attention model, the second vector k may be understood as a key vector of the first self-attention model, and the third vector v may be understood as a value vector of the first self-attention model.

In act A202, a calculation formula in normalizing the first vector and the second vector to obtain a first processing result and multiplying the first processing result by the third vector to obtain the first correlation information is:

$$Attention ( Q , K , V ) = softmax ( \frac{{qk}^{T}}{\sqrt{{d}_{k}}} ) v ,$$

wherein k^{T} is the transposed vector of the second vector (key vector), dₖ is a dimensionality of the key vector, and softmax is a normalization function.

In an exemplary implementation, in act A1, after acquiring gene expression information of a cell line to be tested and structural information of a drug to be tested, the following is further included: performing a dimensionality reduction operation on the gene expression information through a first convolution neural network to obtain dimensionality-reduced gene expression information; and performing a dimensionality reduction operation on the structural information of the drug through second convolution neural network to obtain dimensionality-reduced drug structural information;
in act A201, the multiplying the gene expression information by a first weight matrix to obtain a first vector includes: multiplying the dimensionality-reduced gene expression information by the first weight matrix to obtain the first vector;
in act A201, the multiplying the structural information of the drug by a second weight matrix to obtain a second vector includes: multiplying the dimensionality-reduced drug structural information by the second weight matrix to obtain the second vector; and
in act A201, the multiplying the structural information of the drug by a third weight matrix to obtain a third vector includes: multiplying the dimensionality-reduced drug structural information by the third weight matrix to obtain the third vector.

In an exemplary implementation, a dimensionality of the gene expression information of the cell line to be tested acquired in act A1 is 1*500, a dimensionality of the structural information of the drug is 72*188, a dimensionality of the dimensionality-reduced gene expression information is 1*188, a dimensionality of the dimensionality-reduced drug structural information is 1*188, dimensionalities of the first vector q, the second vector k and the third vector v obtained in act A201 are all 1*188, and a dimensionality of the first correlation information obtained through the formula

$$Attention ( Q , K , V ) = softmax ( \frac{{qk}^{T}}{\sqrt{{d}_{k}}} ) v$$

### in act A202 is 1*188.

In an exemplary implementation, in act A2, the calculating second correlation information between the structural information of the drug to be tested and the gene mutation information based on a second attention model may include:
act A211, multiplying the gene mutation information by a fourth weight matrix to obtain a fourth vector, multiplying the structural information of the drug by a fifth weight matrix to obtain a fifth vector, and multiplying the structural information of the drug by a sixth weight matrix to obtain a sixth vector; and
act A212, normalizing the fourth vector and the fifth vector to obtain a second processing result, and multiplying the second processing result by the sixth vector to obtain the second correlation information.

In an exemplary implementation, in act A212, the normalizing the fourth vector and the fifth vector to obtain a second processing result may include:
transposing the fifth vector to obtain a transposed vector of the fifth vector, multiplying the fourth vector by the transposed vector of the fifth vector to obtain a second product, and dividing the second product by a second constant to obtain a second processing result, the second constant being an arithmetic square root of a dimensionality of the fifth vector.

For example, in act A211, Q1 is set as the gene mutation information, K1 and V1 are set as the structural information of the drug, the fourth weight matrix is set as W^{Q1}, the fifth weight matrix is set as W^{K1}, and the sixth weight matrix is set as W^{V1}, then the gene mutation information Q1 is multiplied by the fourth weight matrix W^{Q1} to obtain the fourth vector q1=Q1*W^{Q1}, the structural information K1 of the drug is multiplied by the fifth weight matrix W^{K1} to obtain the fifth vector k1=K1*W^{K1}, and the structural information V1 of the drug is multiplied by the sixth weight matrix W to obtain the sixth vector v1=V1*W^{V1}. The fourth vector q1 may be understood as a query vector of a second self-attention model, the fifth vector k1 may be understood as a key vector of the second self-attention model, and the sixth vector v1 may be understood as a value vector of the second self-attention model.

In act A222, a calculation formula in normalizing the fourth vector and the fifth vector to obtain a second processing result and multiplying the second processing result by the sixth vector to obtain the second correlation information is:

$$Attention ( Q 1 , K 1 , V 1 ) = softmax ( \frac{q 1 {k}^{T 1}}{\sqrt{{d}_{k 1}}} ) v 1 ,$$

wherein k^{T1} is the transposed vector of the fifth vector (key vector), dₖ is a dimensionality of the key vector, and softmax is a normalization function.

In an exemplary implementation, in act A1, after acquiring gene mutation information of the cell line to be tested and structural information of a drug to be tested, the following is further included: performing a dimensionality reduction operation on the gene mutation information through a third convolution neural network to obtain dimensionality-reduced gene mutation information; and performing a dimensionality reduction operation on the structural information of the drug through a second convolution neural network to obtain dimensionality-reduced drug structural information;
in act A211, the multiplying the gene mutation information by a fourth weight matrix to obtain a fourth vector includes: multiplying the dimensionality-reduced gene mutation information by the fourth weight matrix to obtain the fourth vector;
in act A211, the multiplying the structural information of the drug by a fifth weight matrix to obtain a fifth vector includes: multiplying the dimensionality-reduced drug structural information by the fifth weight matrix to obtain the fifth vector; and
in act A211, the multiplying the structural information of the drug by a sixth weight matrix to obtain a sixth vector includes: multiplying the dimensionality-reduced drug structural information by the sixth weight matrix to obtain the sixth vector.

In an exemplary implementation, a dimensionality of the gene mutation information of the cell line to be tested acquired in act A1 is 1*310, a dimensionality of the structural information of the drug is 72*188, dimensionalities of the dimensionality-reduced gene mutation information and the dimensionality-reduced drug structural information are both 1*188, dimensionalities of the fourth vector q1, the fifth vector k1 and the sixth vector v1 obtained in act A211 are all 1*188, and a dimensionality of the second correlation information obtained through the formula

$$Attention ( Q 1 , K 1 , V 1 ) = softmax ( \frac{q 1 {k}^{T 1}}{\sqrt{{d}_{k 1}}} ) v 1$$

### in act A212 is 1*188.

In an exemplary implementation, in act A1, the acquiring gene expression information of a cell line to be tested may include act A11 to act A14.

Act A11, acquiring raw data of the gene expression information, the raw data of the gene expression information including average values of a plurality of first gene expression features and standard deviations of the plurality of first gene expression features.

Act A12, normalizing the average values of the plurality of first gene expression features to obtain a plurality of normalized expression average values, normalizing the standard deviations of the plurality of first gene expression features to obtain a plurality of normalized expression standard deviations, and inputting the normalized expression standard deviations and the plurality of normalized expression average values into an encoder. Act A13, controlling the encoder to add or subtract the normalized expression standard deviations corresponding to the normalized expression average values to or from a part of the normalized expression average values to obtain a plurality of processed normalized expression average values, and taking another part of unprocessed normalized expression average values and the plurality of processed normalized expression average values as a plurality of encoding input features.

In an exemplary implementation, act A13 may be understood as adding or subtracting the normalized expression standard deviations corresponding to the normalized expression average values to or from a plurality of normalized expression average values at a certain probability.

In an exemplary implementation, in act A13, the controlling the encoder to add a part of the normalized expression average values or to subtract the normalized expression standard deviations corresponding to the normalized expression average values may include: controlling the encoder to add or subtract the normalized expression standard deviations corresponding to the normalized expression average values to or from each of the part of the normalized expression average values. In another exemplary implementation, in act A13, the controlling the encoder to add or subtract the normalized expression standard deviations corresponding to the normalized expression average values to or from a part of the normalized expression average values may include: controlling the encoder to add the normalized expression standard deviations corresponding to the normalized expression average values to a part of the part of the normalized expression average values, and controlling the encoder to subtract the normalized expression standard deviations corresponding to the normalized expression average values from another part of the part of the normalized expression average values.

Act A14, controlling the encoder to encode the plurality of encoding input features to obtain a plurality of second expression features as gene expression information, the number of the plurality of second gene expression features being less than the number of the plurality of first gene expression features.

In an exemplary implementation, the encoder may include an encoding layer which may include an input layer and an output layer;
in act A14, the controlling the encoder to encode the plurality of encoding input features may include:
controlling the encoder to perform the following operation on the plurality of encoding input features to obtain a plurality of second gene expression features: y=s(Wx̅+b), wherein X̅ is the encoding input feature, y is the second gene expression feature, W is a link weight from the input layer to the output layer, b is a deviation of the output layer, and s is a nonlinear function.

In an exemplary implementation, the encoding layer may also include an intermediate hidden layer between the input layer and the output layer, and the input layer, the intermediate hidden layer and the output layer constitute a three-layer neural network with a gradually decreased number of neurons.

In an exemplary implementation, the number of neurons in the input layer is 1500 to 2500, the number of neurons in the intermediate hidden layer is 500 to 1500, and the number of neurons in the output layer is 250 to 750. For example, the number of neurons in the input layer is 2000, the number of neurons in the intermediate hidden layer is 1000, and the number of neurons in the output layer is 500.

In an exemplary implementation, the sensitivity prediction model includes a four-layer neural network with a gradually decreased number of neurons.

In an exemplary implementation, in the four-layer neural network, the number of neurons in a first-layer neural network is 400 to 600, the number of neurons in a second-layer neural network is 100 to 300, the number of neurons in a third-layer neural network is 80 to 120, and the number of neurons in a fourth-layer neural network is 1 to 5. For example, in the four-layer neural network, the number of neurons in the first-layer neural network is 500, the number of neurons in the second-layer neural network is 200, the number of neurons in the third-layer neural network is 100, and the number of neurons in the fourth-layer neural network is 1.

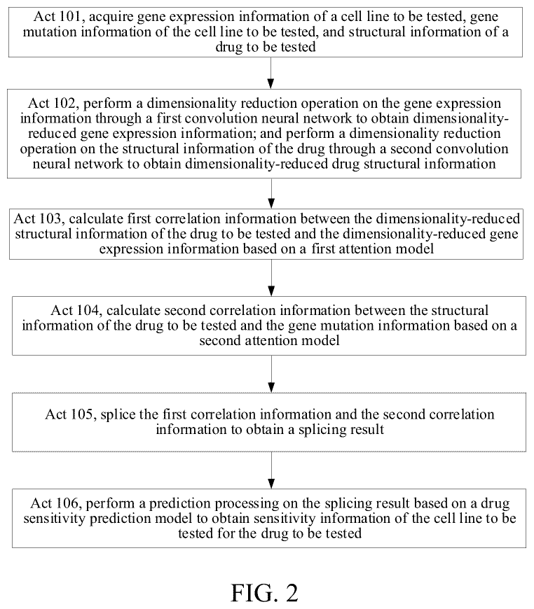

The method for predicting drug sensitivity is described in detail below, as shown in FIG. 2, which may include the following acts.

Act 101, acquiring gene expression information of a cell line to be tested, gene mutation information of the cell line to be tested, and structural information of a drug to be tested.

In an exemplary implementation, the acquiring gene expression information of a cell line to be tested in act 101 may include act B12 to act B14.

Act B11, acquiring raw data of the gene expression information, the raw data of the gene expression information including average values of a plurality of first gene expression features and standard deviations of the plurality of first gene expression features.

Act B12, normalizing the average values of the plurality of first gene expression features to obtain normalized expression average values, normalizing the standard deviations of the plurality of first gene expression features to obtain normalized expression standard deviations, and inputting a plurality of normalized expression standard deviations and a plurality of normalized expression average values into an encoder.

In an embodiment of the present disclosure, the convergence of the model may be increased by normalizing the average values of the plurality of first gene expression features and the standard deviations of the plurality of first gene expression features.

In an exemplary implementation, a calculation formula for normalizing the average value of any first gene expression feature among the average values of the plurality of first gene expression features is Xₙₒᵣₘ=(X−Xₘᵢₙ)/(Xₘₐₓ−Xₘᵢₙ), wherein Xₙₒᵣₘ is the normalized expression average value, X is the average value of the first gene expression feature, Xₘᵢₙ is a minimum value among the average values of the plurality of first gene expression features, and Xₘₐₓ is a maximum value among the average values of the plurality of first gene expression features.

In an exemplary implementation, the normalizing the standard deviation of any first gene expression feature among the standard deviations of the plurality of first gene expression features may include: performing the following operation on the standard deviation of any first gene expression feature among the standard deviations of the plurality of first gene expression features: σₙₒᵣₘ=(σ/X)*Xₙₒᵣₘ, wherein σₙₒᵣₘ is the normalized expression standard deviation, σ is the standard deviation of the first gene expression feature, X is the average value of the first gene expression feature, and Xₙₒᵣₘ is the normalized expression average value. The following operation may be performed on the plurality of first gene expression features and the average values of the plurality of first gene expression features to obtain the standard deviations of the first gene expression features:

$$σ = \sqrt{{\sum }_{i = 1}^{N} {( {X}_{i} - \overline{U} )}^{2} / N} ,$$

wherein σ is the standard deviation of the first gene expression feature, N is the number of first gene expression features, Xᵢ is an i-th first gene expression feature, and Ū is the average value of the plurality of first gene expression features.

Act B13, controlling the encoder to add or subtract the normalized expression standard deviations corresponding to the normalized expression average values to or from a part of the normalized expression average values to obtain a plurality of processed normalized expression average values, and taking another part of unprocessed normalized expression average values and the plurality of processed normalized expression average values as a plurality of encoding input features.

For example, the encoding input feature x=Xₙₒᵣₘ±b*σₙₒᵣₘ, is a deviation of the output layer of the encoding layer in the encoder.

In an exemplary implementation, act B13 may be understood as adding or subtracting the normalized expression standard deviations corresponding to the normalized expression average values to or from a plurality of normalized expression average values at a certain probability.

In an exemplary implementation, in act B13, the controlling the encoder to add or subtract the normalized expression standard deviations corresponding to the normalized expression average values to or from a part of the normalized expression average values may include: controlling the encoder to add or subtract the normalized expression standard deviations corresponding to the normalized expression average values to or from each of the part of the normalized expression average values. In another exemplary implementation, in act B13, the controlling the encoder to add or subtract the normalized expression standard deviations corresponding to the normalized expression average values to or from a part of the normalized expression average values may include: controlling the encoder to add the normalized expression standard deviations corresponding to the normalized expression average values to a part of the part of the normalized expression average values, and controlling the encoder to subtract the normalized expression standard deviations corresponding to the normalized expression average values from another part of the part of the normalized expression average values.

Act B14, controlling the encoder to encode the plurality of encoding input features to obtain a plurality of second gene expression features as gene expression information, the number of the plurality of second gene expression features being less than the number of the plurality of first gene expression features.

In an exemplary implementation, as shown in FIG. 3, the encoder may include an encoding layer which may include an input layer and an output layer; and the controlling the encoder to encode the plurality of encoding input features may include: controlling the encoder to perform the following operation on the plurality of encoding input features to obtain a plurality of second gene expression features: y=s(Wx̅+b), wherein X̅ is the encoding input feature, y is the second gene expression feature, W is a link weight from the input layer to the output layer, b is a deviation of the output layer, and s is a nonlinear function. In an exemplary implementation, the nonlinear function s may be a sigmoid function.

In an exemplary implementation, as shown in FIG. 3, the encoding layer may also include an intermediate hidden layer between the input layer and the output layer, and the input layer, the intermediate hidden layer and the output layer constitute a three-layer neural network with a gradually decreased number of neurons. In an exemplary implementation, the number of neurons in the input layer is 1500 to 2500, the number of neurons in the intermediate hidden layer is 500 to 1500, and the number of neurons in the output layer is 250 to 750. For example, the number of neurons in the input layer is 1000, the number of neurons in the intermediate hidden layer is 1000, and the number of neurons in the output layer is 500. In an embodiment of the present disclosure, the number of layers of neural network in the encoding layer of the encoder and the number of neurons in each layer of neural network may be set according to actual needs at the time of modeling. For example, the number of neurons in the input layer may be set to be greater than 1000 or less than 1000 according to the number of encoding input feature values, the intermediate hidden layer may be set to be of one or more layers, and the number of neurons in the output layer may be set to be less than 500 or greater than 500 according to actual needs. In an embodiment of the present disclosure, the encoder may be an Autoencoder which is a neural network model. In an embodiment of the present disclosure, since there are many features in the acquired gene expression information, encoding a plurality of first gene expression features by an encoder allows for dimensionality reduction, so that the number of second gene expression features output by the output layer is less than the number of first gene expression features, which may save storage space, improve calculation speed and remove redundant features. As shown in FIG. 3, in the encoding layer of the encoder, the number of neurons in the output layer is less than the number of neurons in the input layer.

In an embodiment of the present disclosure, since a dimensionality of the gene expression information is much larger than that of the gene mutation information and of the structural information of the drug, and in addition to the expression average values, the E-MTAB-3610 data set contains expression standard deviation data (the expression level of individual gene is affected by time and space characteristics of expression and by interference, and must be a dynamic level, so the use of standard deviation can better reflect the actual biological significance), the mode of incorporating expression standard deviation on the basis of Autoencoders in an embodiment of the present disclosure may reflect actual biological characteristics.

In an exemplary implementation, the gene expression information of the cell line to be tested and the gene mutation information of the cell line to be tested may be acquired by means of gene detection, and the structural information of the drug to be tested may be acquired from the Genomics of Drug Sensitivity in Cancer (GDSC) database.

The structural information of the drug to be tested is represented by a SMILES structure, in which letters, numbers and special characters are used to represent a molecule. For example, “C” represents a carbon atom, “=” represents a covalent bond between two atoms, a carbon dioxide may be represented as O—C—O, and aspirin may be represented as O═C(C)OC1CCCCC1C(═O)O, wherein the longest SMILES expression of drugs has a total of 188 bits. In the SMILES structural information of 223 anti-tumor drugs analyzed, there are 72 different characters in total. An encoding form of one-hot is considered for processing, i.e., the structural information of each drug may be converted into a one-hot matrix of 72*188. For each drug, the value in an i-th row and a j-th column being 1 means that an i-th symbol appears in a j-th position in the SMILES format, as shown in Table 1:

TABLE 1

| | 188 bits | | | | | |
|---|---|---|---|---|---|---|
| 72 | The first-bit | The second-bit | The third-bit | | The 187th-bit | The 188th-bit |
| characters | character | character | character | . . . | character | character |
| O | 1 | 0 | 0 | . . . | 0 | 0 |
| C | 0 | 0 | 1 | . . . | 0 | 0 |
| . . . | . . . | . . . | . . . | . . . | . . . | . . . |
| H | 0 | 0 | 0 | . . . | 0 | 0 |
| = | 0 | 1 | 0 | . . . | 0 | 0 |

In Table 1, in the SMILES structure of the drug, the first-bit character is O, the second-bit character is =, and the third-bit character is C.

For example, encoding of carbon dioxide O—C—O by one-hot is as shown in Table 2.

TABLE 2

| | 188 bits | | | | | |
|---|---|---|---|---|---|---|
| | The | The | The | The | The | The sixth to |
| 72 | first-bit | second-bit | third-bit | fourth-bit | fifth-bit | 188th-bit |
| characters | character | character | character | character | character | characters |
| O | 1 | 0 | 0 | 0 | 1 | 0 |
| C | 0 | 0 | 1 | 0 | 0 | 0 |
| . . . | 0 | 0 | 0 | 0 | 0 | 0 |
| H | 0 | 0 | 0 | 0 | 0 | 0 |
| = | 0 | 1 | 0 | 1 | 0 | 0 |

Act 102, performing a dimensionality reduction operation on the gene expression information through a first convolution neural network to obtain dimensionality-reduced gene expression information; and performing a dimensionality reduction operation on the structural information of the drug through a second convolution neural network to obtain dimensionality-reduced drug structural information.

In an exemplary implementation, a dimensionality of the gene expression information of the cell line to be tested acquired in act 101 is 1*500, a dimensionality of the structural information of the drug is 72*188, a dimensionality of the gene expression information after dimensionality reduction by the convolution neural network is 1*188, and a dimensionality of the dimensionality-reduced drug structural information is 1*188.

Act 103, calculating first correlation information between the dimensionality-reduced structural information of the drug to be tested and the dimensionality-reduced gene expression information based on a first attention model.

In an exemplary implementation, act 103 may include:
act A01, multiplying the dimensionality-reduced gene expression information by a first weight matrix to obtain a first vector, multiplying the dimensionality-reduced drug structural information by a second weight matrix to obtain a second vector, and multiplying the dimensionality-reduced drug structural information by a third weight matrix to obtain a third vector; and
act A02, normalizing the first vector and the second vector to obtain a first processing result, and multiplying the first processing result by the third vector to obtain the first correlation information.

In an exemplary implementation, in act A02, the normalizing the first vector and the second vector includes: transposing the second vector to obtain a transposed vector of the second vector, multiplying the first vector by the transposed vector of the second vector to obtain a first product, and dividing the first product by a first constant to obtain a first processing result, the first constant being an arithmetic square root of a dimensionality of the second vector.

For example, in act A01, Q is set as the gene expression information, K and V are set as the structural information of the drug, the first weight matrix is set as W^{Q}, the second weight matrix is set as W^{K}, and the third weight matrix is set as W^{V}, then the dimensionality-reduced gene expression information Q is multiplied by the first weight matrix W^{V} to obtain the first vector q=Q*W^{Q}, the dimensionality-reduced drug structural information K is multiplied by the second weight matrix W^{K} to obtain the second vector k=K*W^{K}, and the dimensionality-reduced drug structural information V is multiplied by the third weight matrix W^{V} to obtain the third vector v=V*W^{V}. The first vector q may be understood as a query vector of a first self-attention model, the second vector k may be understood as a key vector of the first self-attention model, and the third vector v may be understood as a value vector of the first self-attention model.

In act A02, a calculation formula in normalizing the first vector and the second vector to obtain a first processing result and multiplying the first processing result by the third vector to obtain the first correlation information is:

$$Attention ( Q , K , V ) = softmax ( \frac{{qk}^{T}}{\sqrt{{d}_{k}}} ) v ,$$

wherein k^{T} is the transposed vector of the second vector (key vector), dₖ is a dimensionality of the key vector, and softmax is a normalization function.

In an exemplary implementation, dimensionalities of the first vector q, the second vector k, and the third vector v obtained in act A01 are all 1*188, and a dimensionality of the first correlation information obtained through the calculation formula

$$Attention ( Q , K , V ) = softmax ( \frac{{qk}^{T}}{\sqrt{{d}_{k}}} ) v$$

### in act A02 is 1*188.

Act 104, calculating second correlation information between the structural information of the drug to be tested and the gene mutation information based on a second attention model.

In an exemplary implementation, act 104 may include:
act A21, performing a dimensionality reduction operation on the gene mutation information through a third convolution neural network to obtain dimensionality-reduced gene mutation information, multiplying the dimensionality-reduced gene mutation information by a fourth weight matrix to obtain a fourth vector, multiplying the dimensionality-reduced drug structural information by a fifth weight matrix to obtain a fifth vector, and multiplying the dimensionality-reduced drug structural information by a sixth weight matrix to obtain a sixth vector; and
act A22, normalizing the fourth vector and the fifth vector to obtain a second processing result, and multiplying the second processing result by the sixth vector to obtain second correlation information.

In an exemplary implementation, in act A22, the normalizing the fourth vector and the fifth vector may include: transposing the fifth vector to obtain a transposed vector of the fifth vector, multiplying the fourth vector by the transposed vector of the fifth vector to obtain a second product, and dividing the second product by a second constant to obtain a second processing result, the second constant being an arithmetic square root of a dimensionality of the fifth vector.

For example, in act A21, Q1 is set as the gene mutation information, K1 and V1 are set as the dimensionality-reduced drug structural information, the fourth weight matrix is set as W^{Q1}, the fifth weight matrix is set as W^{K1}, and the sixth weight matrix is set as W^{V1}, then the dimensionality-reduced gene mutation information Q1 is multiplied by the fourth weight matrix W^{Q1} to obtain the fourth vector q1=Q1*W^{Q1}, the dimensionality-reduced drug structural information K1 is multiplied by the fifth weight matrix W^{K1} to obtain the fifth vector k1=K1*W^{K1}, and the dimensionality-reduced drug structural information V1 is multiplied by the sixth weight matrix W to obtain the sixth vector v1=V1*W^{V1}. The fourth vector q1 may be understood as a query vector of a second self-attention model, the fifth vector k1 may be understood as a key vector of the second self-attention model, and the sixth vector v1 may be understood as a value vector of the second self-attention model.

In act A22, a calculation formula in normalizing the fourth vector and the fifth vector to obtain a second processing result and multiplying the second processing result by the sixth vector to obtain the correlation second information is:

$$Attention ( Q 1 , K 1 , V 1 ) = softmax ( \frac{q 1 {k}^{T 1}}{\sqrt{{d}_{k 1}}} ) v 1 ,$$

wherein k^{T1} is the transposed vector of the fifth vector (key vector), dₖ is a dimensionality of the key vector, and softmax is a normalization function.

In an exemplary implementation, a dimensionality of the gene mutation information of the cell line to be tested acquired in act 101 is 1*310, a dimensionality of the structural information of the drug is 72*188, a dimensionality of the dimensionality-reduced gene mutation information is 1*188, a dimensionality of the dimensionality-reduced drug structural information is 1*188, dimensionalities of the fourth vector q1, the fifth vector k1 and the sixth vector v1 obtained in act A21 are all 1*188, and a dimensionality of the second correlation information obtained through the formula

$$Attention ( Q 1 , K 1 , V 1 ) = softmax ( \frac{q 1 {k}^{T 1}}{\sqrt{{d}_{k 1}}} ) v 1$$

### in act A22 is 1*188.

Act 105, splicing the first correlation information and the second correlation information to obtain a splicing result.

In an exemplary implementation, a splicing result of a 1*376 dimensionality may be obtained after splicing the first correlation information of a 1*188 dimensionality and the second correlation information of a 1*188 dimensionality.

Act 106, performing a prediction processing on the splicing result based on a drug sensitivity prediction model to obtain sensitivity information of the cell line to be tested for the drug to be tested.

In an exemplary implementation, the sensitivity prediction model includes a four-layer neural network with a gradually decreased number of neurons.

In an exemplary implementation, in the four-layer neural network, the number of neurons in a first-layer neural network is 400 to 600, the number of neurons in a second-layer neural network is 100 to 300, the number of neurons in a third-layer neural network is 80 to 120, and the number of neurons in a fourth-layer neural network is 1 to 5. For example, in the four-layer neural network, the number of neurons in the first-layer neural network is 500, the number of neurons in the second-layer neural network is 200, the number of neurons in the third-layer neural network is 100, and the number of neurons in the fourth-layer neural network is 1.

The method for predicting drug sensitivity provided by an embodiment of the present disclosure may predict sensitivity information of a cell line to be tested for a drug to be tested, and the drug to be tested may be a drug for treating tumors or other diseases. In an embodiment of the present disclosure, drug sensitivity information may be IC50 (half maximal inhibitory concentration) or may be log 10 (IC50). In the anti-tumor drug-cell line dose data, GDSC uses IC50 to evaluate the therapeutic effect of an anti-tumor drug. Since IC50 value varies greatly for different cell lines and different drugs, log 10 (IC50) may be used as drug sensitivity information. IC50 is a concentration at which 50% inhibition of growth can be achieved after 72 hours of drug administration to a cell line.

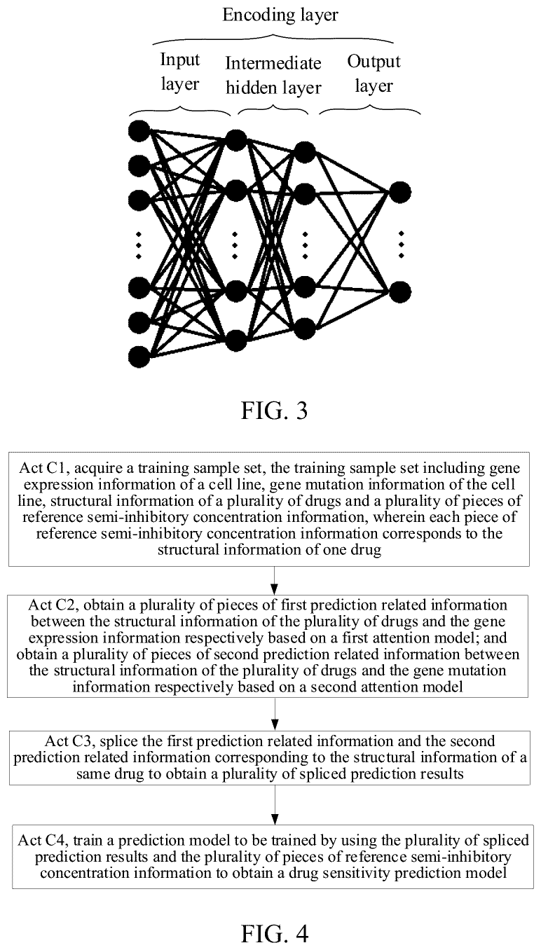

An embodiment of the present disclosure further provides a method for training a drug sensitivity prediction model. As shown in FIG. 4, the method for training a drug sensitivity prediction model provided by an embodiment of the present disclosure may include acts C1-C4:
act C1, acquiring a training sample set, the training sample set including gene expression information of a cell line, gene mutation information of the cell line, structural information of a plurality of drugs and a plurality of pieces of reference semi-inhibitory concentration information, wherein each piece of reference semi-inhibitory concentration information corresponds to the structural information of one drug;
act C2, obtaining a plurality of pieces of first prediction related information between the structural information of the plurality of drugs and the gene expression information respectively based on a first attention model; and obtaining a plurality of pieces of second prediction related information between the structural information of the plurality of drugs and the gene mutation information respectively based on a second attention model;
act C3, splicing the first prediction related information and the second prediction related information corresponding to the structural information of a same drug to obtain a plurality of spliced prediction results; and
act C4, training a prediction model to be trained by using the plurality of spliced prediction results and the plurality of pieces of reference semi-inhibitory concentration information to obtain a drug sensitivity prediction model.

In the method for training a drug sensitivity prediction model provided by an embodiment of the present disclosure, a plurality of pieces of first prediction related information between the structural information of the plurality of drugs to be tested and the gene expression information are obtained through a first attention model, a plurality of pieces of second prediction related information between the structural information of the plurality of drugs to be tested and the gene mutation information are obtained through a second attention model, the first prediction related information and the second prediction related information involving the structural information of a same drug are spliced to obtain a plurality of spliced prediction results, and a prediction model to be trained is trained by using the plurality of spliced prediction results and a plurality of pieces of reference semi-inhibitory concentration information to obtain a drug sensitivity prediction model. Prior to training the prediction model to be trained, prediction related information between the gene expression information, the gene mutation information and the drug structural information is obtained through an attention mechanism. Training the model to be trained according to the prediction related information may improve the prediction effect of the drug sensitivity prediction model.

In an exemplary implementation, the structural information of a plurality of drugs and the plurality of pieces of reference semi-inhibitory concentration information may be acquired from the Genomics of Drug Sensitivity in Cancer (GDSC) database in act C1.

In an exemplary implementation, the gene mutation information of the cell line to be tested acquired in act C1 may be acquired from Genetic Features under the Downloads module in the Genomics of Drug Sensitivity in Cancer (GDSC) database. The gene mutation information is a 310-dimensionality vector, wherein 1 represents the case where a corresponding gene has a mutation and 0 represents the case where a corresponding gene has no mutation.

In an exemplary implementation, the acquiring a training sample set in act C1 may include act C01 to act C04.

Act C01, acquiring raw data of the gene expression information, the raw data of the gene expression information including average values of a plurality of first gene expression features and standard deviations of the plurality of first gene expression features.

In an exemplary implementation, in act C01, original data of the gene expression information of a cell line may be acquired from the Broad Institute Cancer Cell Line Encyclopedia (CCLE) database, and the raw data of the gene expression information of the cell line may be obtained according to the original data of the gene expression information of the cell line. The original data of the gene expression information of the cell line may include a plurality of first gene expression features, and the average values of a plurality of first gene expression features and the standard deviations of the plurality of first gene expression features in the raw data of the gene expression information of the cell line are calculated according to the plurality of first gene expression features.

In an exemplary implementation, the standard deviation σ of the first gene expression features may be calculated through the following formula:

$$σ = \sqrt{{\sum }_{i = 1}^{N} {( {X}_{i} - \overline{U} )}^{2} / N} ,$$

wherein N is the number of first gene expression features, Xᵢ is an i-th first gene expression feature, and Ū is the average value of the plurality of first gene expression features. The average value Ū of the plurality of first gene expression features is obtained by averaging the plurality of first gene expression features.

Act C02, normalizing the average values of the plurality of first gene expression features to obtain a plurality of normalized expression average values, normalizing the standard deviations of the plurality of first gene expression features to obtain a plurality of normalized expression standard deviations, and inputting the plurality of normalized standard deviations and the plurality of normalized expression average values into an encoder.

In an exemplary implementation, in act C02, a calculation formula for normalizing the average value of any first gene expression feature among the average values of the plurality of first gene expression features is Xₙₒᵣₘ=(X−Xₘᵢₙ)/(Xₘₐₓ−Xₘᵢₙ), wherein Xₙₒᵣₘ is the normalized expression average value, X is the average value of the first gene expression feature, Xₘᵢₙ is a minimum value among the average values of the plurality of first gene expression features, and Xₘₐₓ is a maximum value among the average values of the plurality of first gene expression features.

In an exemplary implementation, in act C02, a calculation formula for normalizing the standard deviation of any first gene expression feature among the standard deviations of the plurality of first gene expression features is σₙₒᵣₘ=(σ/X)*Xₙₒᵣₘ, wherein θₙₒᵣₘ is the normalized expression standard deviation, σ is the standard deviation of the first gene expression feature, X is the average value of the first gene expression feature, and Xₙₒᵣₘ is the normalized expression average value.

In an embodiment of the present disclosure, the convergence of the model may be increased by normalizing the average values of the plurality of first gene expression features and the standard deviations of the plurality of first gene expression features.

In an embodiment of the present disclosure, since a dimensionality of the gene expression information is much larger than that of the gene mutation information and of the structural information of the drug, and in addition to the expression average values, the E-MTAB-3610 data set contains expression standard deviation data (the expression level of individual gene is affected by time and space characteristics of expression and by interference, and must be a dynamic level, so the use of standard deviation can better reflect the actual biological significance), the mode of incorporating expression standard deviation on the basis of Autoencoders in an embodiment of the present disclosure may reflect actual biological characteristics.

Act C03, controlling the encoder to add or subtract the normalized expression standard deviations corresponding to the normalized expression average values to or from a part of the normalized expression average values to obtain a plurality of processed normalized expression average values, and taking another part of unprocessed normalized expression average values and the plurality of processed normalized expression average values as a plurality of encoding input features.

For example, the encoding input feature x̅=Xₙₒᵣₘ±b*σₙₒᵣₘ, b is a deviation of the output layer of the encoding layer in the encoder.

In an exemplary implementation, act C03 may be understood as adding or subtracting the normalized expression standard deviations corresponding to the normalized expression average values to or from a plurality of normalized expression average values at a certain probability.

In an exemplary implementation, in act C03, the controlling the encoder to add or subtract the normalized expression standard deviations corresponding to the normalized expression average values to or from a part of the normalized expression average values may include: controlling the encoder to add or subtract the normalized expression standard deviations corresponding to the normalized expression average values to or from each of the part of the normalized expression average values. In another exemplary implementation, in act C03, the controlling the encoder to add or subtract the normalized expression standard deviations corresponding to the normalized expression average values to or from a part of the normalized expression average values may include: controlling the encoder to add the normalized expression standard deviations corresponding to the normalized expression average values to a part of the part of the normalized expression average values, and controlling the encoder to subtract the normalized expression standard deviations corresponding to the normalized expression average values from another part of the part of the normalized expression average values.

Act C04, controlling the encoder to encode the plurality of encoding input features to obtain gene expression information, wherein the gene expression information includes a plurality of second gene expression features, and the number of the plurality of second gene expression features is less than the number of the plurality of first gene expression features.

In an exemplary implementation, as shown in FIG. 3, the encoder includes an encoding layer which includes an input layer and an output layer; and the controlling the encoder to encode the plurality of encoding input features may include: controlling the encoder to perform the following operation on the plurality of encoding input features to obtain a plurality of second gene expression features: y=s(Wx̅+b), wherein X̅ is the encoding input feature, y is the second gene expression feature, W is a link weight from the input layer to the output layer, b is a deviation of the output layer, and s is a nonlinear function. In an exemplary implementation, the nonlinear function s may be a sigmoid function.

In an exemplary implementation, as shown in FIG. 3, the encoding layer may also include an intermediate hidden layer between the input layer and the output layer, and the input layer, the intermediate hidden layer and the output layer constitute a three-layer neural network with a gradually decreased number of neurons. In an exemplary implementation, the number of neurons in the input layer is 1500 to 2500, the number of neurons in the intermediate hidden layer is 500 to 1500, and the number of neurons in the output layer is 250 to 750. For example, the number of neurons in the input layer is 2000, the number of neurons in the intermediate hidden layer is 1000, and the number of neurons in the output layer is 500. In an embodiment of the present disclosure, the number of layers of neural network in the encoding layer of the encoder and the number of neurons in each layer of neural network may be set according to actual needs at the time of modeling. For example, the number of neurons in the input layer may be set to be greater than 1000 or less than 1000 according to the number of encoding input feature values, the intermediate hidden layer may be set to be of one or more layers, and the number of neurons in the output layer may be set to be less than 500 or greater than 500 according to actual needs. In an embodiment of the present disclosure, the encoder may be an Autoencoder which is a neural network model. In an embodiment of the present disclosure, since there are many features in the acquired gene expression information, encoding a plurality of first gene expression features by an encoder allows for dimensionality reduction, so that the number of second gene expression features output by the output layer is less than the number of first gene expression features, which may save storage space, improve calculation speed and remove redundant features. As shown in FIG. 3, in the encoding layer of the encoder, the number of neurons in the output layer is less than the number of neurons in the input layer.

In an exemplary implementation, the structural information of any one of the drugs to be tested may be represented in a manner described in Table 1 above.

In an exemplary implementation, prior to performing inputting the plurality of normalized standard deviations and the plurality of normalized expression average values into the encoder in act C02, the following is further included.

Act C0, acquiring a training sample set of the first gene expression features, the training sample set of the first gene expression features including average value samples of a plurality of first gene expression features and corresponding standard deviation samples of the plurality of first gene expression features, and training the encoder according to the average value samples of the plurality of first gene expression features and the standard deviation samples of the plurality of first gene expression features to obtain a link weight W, a deviation b of the output layer and a nonlinear function s.

Act C0 is performed prior to inputting the plurality of normalized standard deviations and the plurality of normalized expression average values into the encoder, and may be, but not limited to, performed in act C02 or prior to act C01.

In an exemplary implementation, the acquiring a training sample set of the first gene expression features may include: acquiring original data of the training sample set of the first gene expression features from a Broad Institute Cancer Cell Line Encyclopedia (CCLE) database, and calculating raw data of the training sample set of the first gene expression features according to the original data of the training sample set of the first gene expression features. The original data of the training sample set of the first gene expression features may include training samples of the plurality of first gene expression features, the raw data of the training sample set of the first gene expression features may include average values of the plurality of first gene expression features of the plurality of first gene expression features and standard deviations of the plurality of first gene expression features, and the average values of the plurality of first gene expression features and the standard deviations of the plurality of first gene expression features are calculated according to the plurality of first gene expression features.

In an exemplary implementation, the training the encoder according to the average value samples of the plurality of first gene expression features and the standard deviation samples of the plurality of first gene expression features in act C0 may include act D0: inputting a plurality of spliced prediction results into an encoder to be trained for a plurality of times through multiple iterations, and optimizing the encoder to be trained according to a result of each iteration to obtain a trained encoder.

In an exemplary implementation, the encoder further includes a decoding layer; the inputting a plurality of spliced prediction results into an encoder to be trained for a plurality of times through multiple iterations, and optimizing the encoder to be trained according to a result of each iteration in act D0 may include act D01 to act D05:
act D01, normalizing the average value samples of the plurality of first gene expression features to obtain normalized expression average value samples, normalizing the standard deviation samples of the plurality of first gene expression features to obtain normalized expression standard deviation samples, and inputting the plurality of normalized standard deviation samples and the plurality of normalized expression average value samples into the encoder to be trained;
act D02, controlling the encoder to be trained to add or subtract the expression standard deviation samples corresponding to the normalized expression average value samples to or from a part of the normalized expression average value samples to obtain a plurality of processed normalized expression average value samples, and taking another part of unprocessed normalized expression average value samples and the plurality of processed normalized expression average value samples as a plurality of encoding input feature samples;
act D03, controlling the encoder to be trained to encode the plurality of encoding input feature samples to obtain a plurality of second gene expression feature samples as gene expression information samples, the number of the plurality of second gene expression feature samples being less than the number of the plurality of first gene expression feature samples;
act D04, inputting the gene expression information samples into the decoding layer to obtain decoding information; and
act D05, calculating a loss value according to the decoding information and the average value samples of the plurality of first gene expression features, optimizing the encoder according to the loss value, using the optimized encoder as an encoder to be trained in a next iteration, and continuously controlling the encoder to be trained to add or subtract the expression standard deviation samples corresponding to the normalized expression average value samples to or from a part of the normalized expression average value samples.

In an exemplary implementation, act D02 may be understood as adding or subtracting the normalized expression standard deviation samples corresponding to the normalized expression average value samples to or from a plurality of normalized expression average value samples at a certain probability.

In an exemplary implementation, in act D02, the controlling the encoder to add or subtract the normalized expression standard deviation samples corresponding to the normalized expression average value samples to or from a part of the normalized expression average value samples may include: controlling the encoder to add or subtract the normalized expression standard deviation samples corresponding to the normalized expression average value samples to or from each of the part of the normalized expression average value samples. In another exemplary implementation, in act D02, the controlling the encoder to add or subtract the normalized expression standard deviation samples corresponding to the normalized expression average value samples to or from a part of the normalized expression average value samples may include: controlling the encoder to add the normalized expression standard deviation samples corresponding to the normalized expression average value samples to a part of the part of the normalized expression average value samples, and controlling the encoder to subtract the normalized expression standard deviation samples corresponding to the normalized expression average value samples from another part of the part of the normalized expression average value samples.

In an exemplary implementation, in act D04, the inputting the gene expression information into the decoding layer to obtain decoding information may include: controlling the encoder to be trained to perform the following operation on the gene expression information to obtain the decoding information: z=s (W′y+b′), wherein s is a nonlinear function, W′ is a link weight of the decoding layer, b′ is a deviation of the decoding layer, y is a feature value in the gene expression information, and z is a feature value of the decoding information.

In act D05, the calculating a loss value according to the decoding information and the average value samples of the plurality of first gene expression features may include: controlling the encoder to be trained to perform the following operation according to the average value samples of the plurality of first gene expression features and the decoding information to obtain the loss value: L(x,z)=∥x−z∥², wherein L (x,z) is a loss function, x is a feature value in the average value samples of the first gene expression features, and z is a feature value of the decoding information.

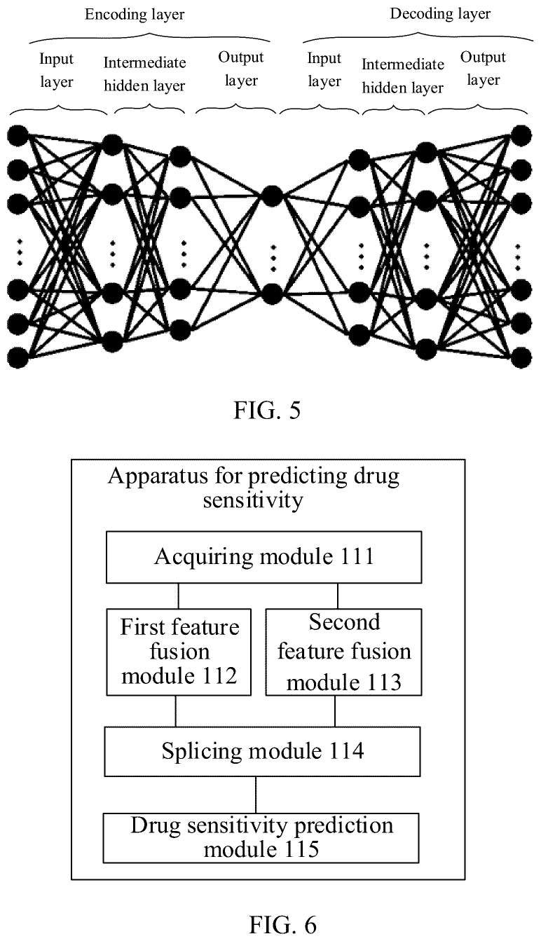

In an embodiment of the present disclosure, as shown in FIG. 5, the decoding layer and the encoding layer may be symmetrical, and neurons of the output layer of the encoding layer may be used as neurons of the input layer of the decoding layer. Information output from the decoding layer is equivalent to verification of the encoding effect of the encoding layer.

In an exemplary implementation, as shown in FIG. 5, the decoding layer and the encoding layer in the encoder are each a three-layer neural network.

In an exemplary implementation, an input layer, an intermediate hidden layer, and an output layer in the encoding layer in the encoder constitute a three-layer neural network with a gradually decreased number of neurons. In an exemplary implementation, in the encoding layer, the number of neurons in the input layer is 1500 to 2500, the number of neurons in the intermediate hidden layer is 500 to 1500, and the number of neurons in the output layer is 250 to 750. For example, in the encoding layer, the number of neurons in the input layer is 2000, the number of neurons in the intermediate hidden layer is 1000, and the number of neurons in the output layer is 500.

In an exemplary implementation, an input layer, an intermediate hidden layer and an output layer in the decoding layer constitute a three-layer neural network with a gradually increased number of neurons. In an exemplary implementation, in the decoding layer, the number of neurons in the input layer is 250 to 750, the number of neurons in the intermediate hidden layer is 500 to 1500, and the number of neurons in the output layer is 1500 to 2500. For example, in the decoding layer, the number of neurons in the input layer is 500, the number of neurons in the intermediate hidden layer is 1000, and the number of neurons in the output layer is 2000.

In an embodiment of the present disclosure, the encoder shown in FIG. 5 may be an Autoencoder.

In an exemplary implementation, in act C2, the obtaining a plurality of pieces of first prediction related information between the structural information of the plurality of drugs and the gene expression information respectively based on a first attention model may include:
M11, multiplying the gene expression information by a first weight matrix to obtain a first vector, multiplying the structural information of the plurality of drugs by a corresponding second weight matrix respectively to obtain a plurality of second vectors, and multiplying the structural information of the plurality of drugs by a corresponding third weight matrix respectively to obtain a plurality of third vectors;
M12, normalizing the first vector and the second vector corresponding to the structural information of a same drug to obtain a plurality of first processing results; and
M13, multiplying the first processing result by the third vector corresponding to the structural information of a same drug to obtain a plurality of pieces of first correlation information.

In an exemplary implementation, in act C2, prior to obtaining a plurality of pieces of first prediction related information between the structural information of the plurality of drugs and the gene expression information respectively based on a first attention model, the following may further be included: training the first attention model using the structural information of the plurality of drugs and the gene expression information to obtain a first weight matrix, a second weight matrix and a third weight matrix.

In an exemplary implementation, in act C2, the obtaining a plurality of pieces of second prediction related information between the structural information of the plurality of drugs and the gene mutation information respectively based on a second attention model may include:
act M21, multiplying the gene mutation information by a fourth weight matrix to obtain a fourth vector, multiplying the structural information of the plurality of drugs by a corresponding fifth weight matrix respectively to obtain a plurality of fifth vectors, and multiplying the structural information of the plurality of drugs by a corresponding sixth weight matrix respectively to obtain a plurality of sixth vectors;
act M22, normalizing the fourth vector and the fifth vector corresponding to the structural information of a same drug to obtain a plurality of second processing results; and
act M23, multiplying the second processing result by the sixth vector corresponding to the structural information of a same drug to obtain a plurality of pieces of second correlation information.

In an exemplary implementation, in act C2, prior to obtaining a plurality of pieces of second prediction related information between the structural information of the plurality of drugs and the gene mutation information respectively based on a second attention model, the following may further be included: training the second attention model using the structural information of the plurality of drugs and the gene expression information to obtain a fourth weight matrix, a fifth weight matrix and a sixth weight matrix.

In an exemplary implementation, in act C4, the training a prediction model to be trained by using the plurality of spliced prediction results and the plurality of pieces of reference semi-inhibitory concentration information to obtain a drug sensitivity prediction model may include: training the prediction model to be trained in a multi-iteration manner for multiple times according to the plurality of spliced prediction results and the plurality of pieces of reference semi-inhibitory concentration information to obtain the drug sensitivity prediction model.

During each iteration, the plurality of spliced prediction results are input into the drug sensitivity model to be trained to obtain a plurality of pieces of predicted semi-inhibitory concentration information, sensitivity loss information is obtained according to the plurality of pieces of predicted semi-inhibitory concentration information and the plurality of pieces of reference semi-inhibitory concentration information, the prediction model to be trained is optimized according to the sensitivity loss information, and the optimized model is used as a prediction model to be trained in a next iteration. Alternatively, during each iteration, the plurality of spliced prediction results are input into the prediction model to be trained in batches to obtain a plurality of pieces of predicted semi-inhibitory concentration information of a current batch, sensitivity loss information of the current batch is obtained according to the plurality of pieces of predicted semi-inhibitory concentration information of the current batch and a plurality of pieces of reference semi-inhibitory concentration information corresponding to the current batch, the prediction model to be obtained is optimized according to the sensitivity loss information of the current batch, and the optimized model is used as a prediction model to be trained for a next batch or a next iteration, wherein when the current batch is a last batch, the optimized model is used as the prediction model to be trained for a next iteration, and when the current batch is not the last batch, the optimized model is used as the prediction model to be trained for a next batch.

In an exemplary implementation, the obtaining sensitivity loss information of the current batch according to the plurality of pieces of predicted semi-inhibitory concentration information of the current batch and a plurality of pieces of reference semi-inhibitory concentration information corresponding to the current batch may include:

performing the following operation on the plurality of pieces of predicted semi-inhibitory concentration information of the current batch and the plurality of pieces of reference semi-inhibitory concentration information corresponding to the current batch to obtain the sensitivity loss information of the current batch:

$$RSME = \sqrt{{\sum }_{i = 1}^{N} {( {y}_{i} - \overline{{y}_{i}} )}^{2} / N} ,$$

wherein y̅ᵢ is the predicted semi-inhibitory concentration information, yᵢ is the reference semi-inhibitory concentration information, RSME is the sensitivity loss information of the current batch, and N is the number in the current batch.

In an exemplary implementation, in act C4, prior to training a prediction model to be trained by using the plurality of spliced prediction results and the plurality of pieces of reference semi-inhibitory concentration information, the following may further be included: setting parameters of the prediction model to be trained.

The parameters of the prediction model to be trained may include: an optimizer being set to SGD, a batch magnitude N being set to 32, the number of iterations being set to 100, and a discard probability being set to 0.001. Among them, SGD is Stochastic Gradient Descent.

In an exemplary implementation, the parameters of the prediction model to be trained may further include: the number of layers of neural network of the prediction model to be trained, and the number of neurons in each layer of neural network;
prior to training a prediction model to be trained by using the plurality of spliced prediction results and the plurality of pieces of reference semi-inhibitory concentration information, the following is further included: establishing a prediction model to be trained according to the parameters of the prediction model to be trained.

In an exemplary implementation, the number of layers of neural network of the prediction model to be trained is four, and the numbers of neurons in the four layers of neural network decrease sequentially, the number of neurons in a first-layer neural network is 400 to 600, the number of neurons in a second-layer neural network is 100 to 300, the number of neurons in a third-layer neural network is 80 to 120, and the number of neurons in a fourth-layer neural network is 1 to 5.

An embodiment of the present disclosure further provides an apparatus for predicting drug sensitivity, as shown in FIG. 6, which may include:
an acquiring module 111, configured to acquire gene expression information of a cell line to be tested, gene mutation information of the cell line to be tested, and structural information of a drug to be tested;
a first feature fusion module 112, configured to calculate first correlation information between the structural information of the drug to be tested and the gene expression information based on a first attention model;
a second feature fusion module 113, configured to calculate second correlation information between the structural information of the drug to be tested and the gene mutation information based on a second attention model;
a splicing module 114, configured to splice the first correlation information and the second correlation information to obtain a splicing result; and
a drug sensitivity prediction module 115, configured to perform a prediction processing on the splicing result based on a drug sensitivity prediction model to obtain sensitivity information of the cell line to be tested for the drug to be tested.

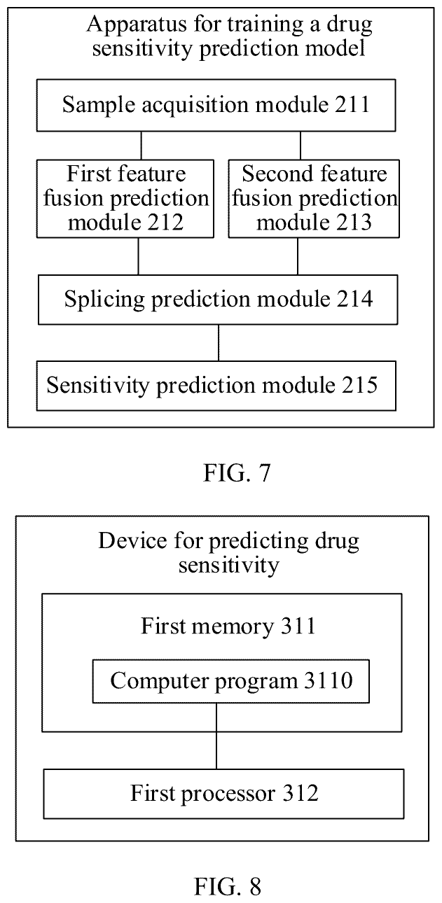

An embodiment of the present disclosure further provides an apparatus for training a drug sensitivity prediction model, as shown in FIG. 7, which may include:
a sample acquisition module 211, configured to acquire a training sample set, the training sample set including gene expression information of a cell line, gene mutation information of the cell line, structural information of a plurality of drugs and a plurality of pieces of reference semi-inhibitory concentration information, wherein each piece of reference semi-inhibitory concentration information corresponds to the structural information of one of the drugs;
a first feature fusion prediction module 212, configured to obtain a plurality of pieces of first prediction related information between the structural information of the plurality of drugs and the gene expression information respectively based on a first attention model;
a second feature fusion prediction module 213, configured to obtain a plurality of pieces of second prediction related information between the structural information of the plurality of drugs and the gene mutation information respectively based on a second attention model;
a splicing prediction module 214, configured to splice the first prediction related information and the second prediction related information involving the structural information of a same drug to obtain a plurality of spliced prediction results; and
a sensitivity prediction module 215, configured to train a prediction model to be trained by using the plurality of spliced prediction results and the plurality of pieces of reference semi-inhibitory concentration information to obtain a drug sensitivity prediction model.

An embodiment of the present disclosure further provides a non-transitory computer-readable storage medium, the storage medium being configured to store computer program instructions, wherein when the computer program instructions are run, the method for predicting drug sensitivity according to any one of the aforementioned embodiments is implemented.

An embodiment of the present disclosure further provides a non-transitory computer-readable storage medium, the storage medium being configured to store computer program instructions, wherein when the computer program instructions are run, the method for training a drug sensitivity prediction model according to any one of the aforementioned embodiments is implemented.

An embodiment of the present disclosure further provides a device for predicting drug sensitivity, as shown in FIG. 8, which may include a first memory 311, a first processor 312, and a computer program 3110 stored on the first memory 311 and runnable on the first processor 312, to perform:
acquiring gene expression information of a cell line to be tested, gene mutation information of the cell line to be tested, and structural information of a drug to be tested;
calculating first correlation information between the structural information of the drug to be tested and the gene expression information based on a first attention model; and calculating second correlation information between the structural information of the drug to be tested and the gene mutation information based on a second attention model;
splicing the first correlation information and the second correlation information to obtain a splicing result; and
performing a prediction processing on the splicing result based on a drug sensitivity prediction model to obtain sensitivity information of the cell line to be tested for the drug to be tested.

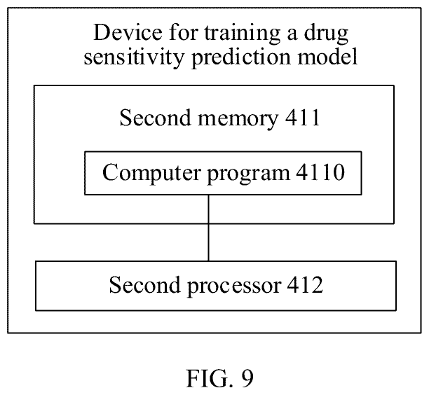

An embodiment of the present disclosure further provides a device for training a drug sensitivity prediction model, as shown in FIG. 9, which may include a second memory 411, a second processor 412, and a computer program 4110 stored on the second memory 411 and runnable on the second processor 412, to perform:
acquiring a training sample set, the training sample set including gene expression information of a cell line, gene mutation information of the cell line, structural information of a plurality of drugs and a plurality of pieces of reference semi-inhibitory concentration information, wherein each piece of reference semi-inhibitory concentration information corresponds to the structural information of one of the drugs;
obtaining a plurality of pieces of first prediction related information between the structural information of the plurality of drugs and the gene expression information respectively based on a first attention model; and obtaining a plurality of pieces of second prediction related information between the structural information of the plurality of drugs and the gene mutation information respectively based on a second attention model;
splicing the first prediction related information and the second prediction related information involving the structural information of a same drug to obtain a plurality of spliced prediction results; and training a prediction model to be trained by using the plurality of spliced prediction results and the plurality of pieces of reference semi-inhibitory concentration information to obtain a drug sensitivity prediction model.

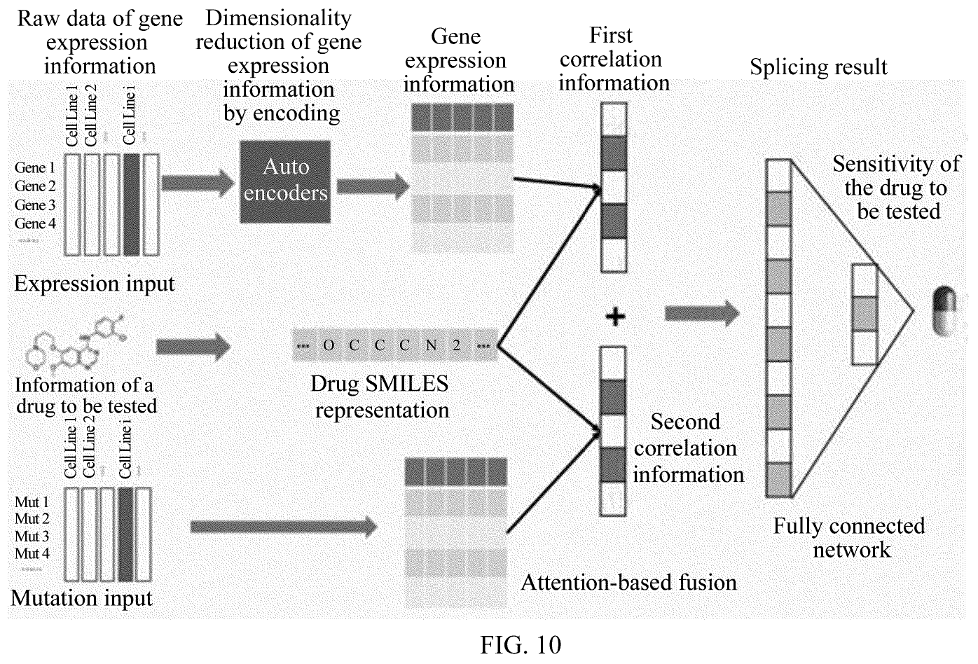

In the method for predicting drug sensitivity provided by an embodiment of the present disclosure, SMILES structural information of a drug is fused with gene expression information to obtain first correlation information, the SMILES structural information of the drug is fused with gene mutation information to obtain second correlation information, the first correlation information and the second correlation information are spliced to obtain a splicing result, and the splicing result is input into a drug sensitivity prediction model of a fully connected network. The schematic diagram of a logical structure of drug sensitivity prediction is shown in FIG. 10.

In the drug sensitivity prediction and model training method, storage medium and device, and method for predicting drug sensitivity provided by the embodiments of the present disclosure, first correlation information between structural information of a drug to be tested and gene expression information is obtained through a first attention model, second correlation information between the structural information of the drug to be tested and gene mutation information is obtained through a second attention model, the first correlation information and the second correlation information are spliced to obtain a splicing result, and the splicing result is processed through a drug sensitivity prediction model to obtain sensitivity information of a cell line to be tested for the drug to be tested. Prior to the prediction through the drug sensitivity prediction model, correlation information between the gene expression information, the gene mutation information and the structural information of the drug is obtained through an attention mechanism, and prediction of drug sensitivity is performed according to the correlation information, which may improve prediction effect of the drug sensitivity prediction model, overcoming the defect of poor effect in drug sensitivity prediction.

The drawings of the embodiments of the present disclosure only involve structures involved in the embodiments of the present disclosure, and for other structures, reference may be made to usual designs.

The embodiments of the present disclosure, that is, features in the embodiments, may be combined with each other to obtain new embodiments if there is no conflict.

Although the implementations disclosed in the embodiments of the present disclosure are described above, the described contents are only implementations used for facilitating understanding of the embodiments of the present disclosure, which are not intended to limit the embodiments of the present disclosure. Any person skilled in the art to which the embodiments of the present disclosure pertain may make any modifications and variations in forms and details of implementation without departing from the spirit and scope disclosed in the embodiments of the present disclosure. Nevertheless, the scope of patent protection of the embodiments of the present disclosure shall still be subject to the scope defined by the appended claims.

1. A method for predicting drug sensitivity, comprising:
acquiring gene expression information of a cell line to be tested, gene mutation information of the cell line to be tested, and structural information of a drug to be tested;
calculating first correlation information between the structural information of the drug to be tested and the gene expression information based on a first attention model; and calculating second correlation information between the structural information of the drug to be tested and the gene mutation information based on a second attention model;
splicing the first correlation information and the second correlation information to obtain a splicing result; and
performing a prediction processing on the splicing result based on a drug sensitivity prediction model to obtain sensitivity information of the cell line to be tested for the drug to be tested.

2. The method for predicting drug sensitivity according to claim 1, wherein the calculating first correlation information between the structural information of the drug to be tested and the gene expression information based on a first attention model comprises:
multiplying the gene expression information by a first weight matrix to obtain a first vector, multiplying the structural information of the drug by a second weight matrix to obtain a second vector, and multiplying the structural information of the drug by a third weight matrix to obtain a third vector; and
normalizing the first vector and the second vector to obtain a first processing result, and multiplying the first processing result by the third vector to obtain the first correlation information.

3. The method for predicting drug sensitivity according to claim 2, wherein the normalizing the first vector and the second vector to obtain a first processing result comprises:
transposing the second vector to obtain a transposed vector of the second vector, multiplying the first vector by the transposed vector of the second vector to obtain a first product, and dividing the first product by a first constant to obtain a first processing result, the first constant being an arithmetic square root of a dimensionality of the second vector.

4. The method for predicting drug sensitivity according to claim 2, wherein after acquiring gene expression information of a cell line to be tested and structural information of a drug to be tested, the method further comprises: performing a dimensionality reduction operation on the gene expression information through a first convolution neural network to obtain dimensionality-reduced gene expression information; and
performing a dimensionality reduction operation on the structural information of the drug through a second convolution neural network to obtain dimensionality-reduced drug structural information;
the multiplying the gene expression information by a first weight matrix to obtain a first vector comprises: multiplying the dimensionality-reduced gene expression information by the first weight matrix to obtain the first vector;
the multiplying the structural information of the drug by a second weight matrix to obtain a second vector comprises: multiplying the dimensionality-reduced drug structural information by the second weight matrix to obtain the second vector; and
the multiplying the structural information of the drug by a third weight matrix to obtain a third vector comprises: multiplying the dimensionality-reduced drug structural information by the third weight matrix to obtain the third vector.

5. The method for predicting drug sensitivity according to claim 1, wherein the calculating second correlation information between the structural information of the drug to be tested and the gene mutation information based on a second attention model comprises:
multiplying the gene mutation information by a fourth weight matrix to obtain a fourth vector, multiplying the structural information of the drug by a fifth weight matrix to obtain a fifth vector, and multiplying the structural information of the drug by a sixth weight matrix to obtain a sixth vector; and
normalizing the fourth vector and the fifth vector to obtain a second processing result, and multiplying the second processing result by the sixth vector to obtain the second correlation information.

6. The method for predicting drug sensitivity according to claim 5, wherein the normalizing the fourth vector and the fifth vector to obtain a second processing result comprises:
transposing the fifth vector to obtain a transposed vector of the fifth vector, multiplying the fourth vector by the transposed vector of the fifth vector to obtain a second product, and dividing the second product by a second constant to obtain a second processing result, the second constant being an arithmetic square root of a dimensionality of the fifth vector.

7. The method for predicting drug sensitivity according to claim 5, wherein after acquiring gene mutation information of the cell line to be tested and structural information of a drug to be tested, the method further comprises: performing a dimensionality reduction operation on the gene mutation information through a third convolution neural network to obtain dimensionality-reduced gene mutation information; and
performing a dimensionality reduction operation on the structural information of the drug through a second convolution neural network to obtain dimensionality-reduced drug structural information;
the multiplying the gene mutation information by a fourth weight matrix to obtain a fourth vector comprises: multiplying the dimensionality-reduced gene mutation information by the fourth weight matrix to obtain the fourth vector;
the multiplying the structural information of the drug by a fifth weight matrix to obtain a fifth vector comprises: multiplying the dimensionality-reduced drug structural information by the fifth weight matrix to obtain the fifth vector; and
the multiplying the structural information of the drug by a sixth weight matrix to obtain a sixth vector comprises: multiplying the dimensionality-reduced drug structural information by the sixth weight matrix to obtain the sixth vector.

8. The method for predicting drug sensitivity according to claim 1, wherein the acquiring gene expression information of a cell line to be tested comprises:
acquiring raw data of the gene expression information, the raw data of the gene expression information comprising average values of a plurality of first gene expression features and standard deviations of the plurality of first gene expression features;
normalizing the average values of the plurality of first gene expression features to obtain a plurality of normalized expression average values, normalizing the standard deviations of the plurality of first gene expression features to obtain a plurality of normalized expression standard deviations, and inputting the plurality of normalized expression standard deviations and the plurality of normalized expression average values into an encoder;
controlling the encoder to add or subtract the normalized expression standard deviations corresponding to the normalized expression average values to or from a part of the normalized expression average values to obtain a plurality of processed normalized expression average values, and taking another part of unprocessed normalized expression average values and the plurality of processed normalized expression average values as a plurality of encoding input features; and controlling the encoder to encode the plurality of encoding input features to obtain a plurality of second gene expression features as the gene expression information, the number of the plurality of second gene expression features being less than the number of the plurality of first gene expression features.

9. The method for predicting drug sensitivity according to claim 8, wherein the encoder comprises an encoding layer, and the encoding layer comprises an input layer and an output layer;
the controlling the encoder to encode the plurality of encoding input features comprises:
controlling the encoder to perform the following operation on the plurality of encoding input features to obtain a plurality of second gene expression features: y=s(Wx̅+b), wherein X̅ is the encoding input feature, y is the second gene expression feature, W is a link weight from the input layer to the output layer, b is a deviation of the output layer, and s is a nonlinear function.

10. The method for predicting drug sensitivity according to claim 9, wherein the encoding layer further comprises an intermediate hidden layer between the input layer and the output layer, and the input layer, the intermediate hidden layer and the output layer constitute a three-layer neural network with a gradually decreased number of neurons.

11. The method for predicting drug sensitivity according to claim 1, wherein
the sensitivity prediction model comprises a four-layer neural network with a gradually decreased number of neurons.

12. A method for training a drug sensitivity prediction model, comprising:
acquiring a training sample set, the training sample set comprising gene expression information of a cell line, gene mutation information of the cell line, structural information of a plurality of drugs and a plurality of pieces of reference semi-inhibitory concentration information, wherein each piece of reference semi-inhibitory concentration information corresponds to the structural information of one drug;
obtaining a plurality of pieces of first prediction related information between the structural information of the plurality of drugs and the gene expression information respectively based on a first attention model; and obtaining a plurality of pieces of second prediction related information between the structural information of the plurality of drugs and the gene mutation information respectively based on a second attention model;
splicing the first prediction related information and the second prediction related information corresponding to the structural information of a same drug to obtain a plurality of spliced prediction results; and
training a prediction model to be trained by using the plurality of spliced prediction results and the plurality of pieces of reference semi-inhibitory concentration information to obtain a drug sensitivity prediction model.

13. The method for training a drug sensitivity prediction model according to claim 12, wherein the training a prediction model to be trained by using the plurality of spliced prediction results and the plurality of pieces of reference semi-inhibitory concentration information to obtain a drug sensitivity prediction model comprises:
training the prediction model to be trained in a multi-iteration manner for multiple times according to the plurality of spliced prediction results and the plurality of pieces of reference semi-inhibitory concentration information to obtain the drug sensitivity prediction model;
wherein during each iteration, the plurality of spliced prediction results are input into the drug sensitivity model to be trained to obtain a plurality of pieces of predicted semi-inhibitory concentration information, sensitivity loss information is obtained according to the plurality of pieces of predicted semi-inhibitory concentration information and the plurality of pieces of reference semi-inhibitory concentration information, the prediction model to be trained is optimized according to the sensitivity loss information, and the optimized model is used as a prediction model to be trained in a next iteration; or
during each iteration, the plurality of spliced prediction results are input into the prediction model to be trained in batches to obtain a plurality of pieces of predicted semi-inhibitory concentration information of a current batch, sensitivity loss information of the current batch is obtained according to the plurality of pieces of predicted semi-inhibitory concentration information of the current batch and a plurality of pieces of reference semi-inhibitory concentration information corresponding to the current batch, the prediction model to be obtained is optimized according to the sensitivity loss information of the current batch, and the optimized model is used as a prediction model to be trained for a next batch or a next iteration.

14. The method for training a drug sensitivity prediction model according to claim 12, wherein the obtaining a plurality of pieces of first prediction related information between the structural information of the plurality of drugs and the gene expression information respectively based on a first attention model comprises:
multiplying the gene expression information by a first weight matrix to obtain a first vector, multiplying the structural information of the plurality of drugs by a corresponding second weight matrix respectively to obtain a plurality of second vectors, and multiplying the structural information of the plurality of drugs by a corresponding third weight matrix respectively to obtain a plurality of third vectors;
normalizing the first vector and the second vector corresponding to the structural information of a same drug to obtain a plurality of first processing results; and
multiplying the first processing result by the third vector corresponding to the structural information of a same drug to obtain the plurality of pieces of first correlation information.

15. The method for training a drug sensitivity prediction model according to claim 14, wherein prior to the obtaining a plurality of pieces of first prediction related information between the structural information of the plurality of drugs and the gene expression information respectively based on a first attention model, the method further comprises:
training the first attention model using the structural information of the plurality of drugs and the gene expression information to obtain a first weight matrix, a second weight matrix and a third weight matrix.

16. The method for training a drug sensitivity prediction model according to claim 12, wherein the obtaining a plurality of pieces of second prediction related information between the structural information of the plurality of drugs and the gene mutation information respectively based on a second attention model comprises:
multiplying the gene mutation information by a fourth weight matrix to obtain a fourth vector, multiplying the structural information of the plurality of drugs by a corresponding fifth weight matrix respectively to obtain a plurality of fifth vectors, and multiplying the structural information of the plurality of drugs by a corresponding sixth weight matrix respectively to obtain a plurality of sixth vectors;
normalizing the fourth vector and the fifth vector corresponding to the structural information of a same drug to obtain a plurality of second processing results; and
multiplying the second processing result by the sixth vector corresponding to the structural information of a same drug to obtain the plurality of pieces of second correlation information.

17. The method for training a drug sensitivity prediction model according to claim 16, wherein prior to the obtaining a plurality of pieces of second prediction related information between the structural information of the plurality of drugs and the gene mutation information respectively based on a second attention model, the method further comprises:
training the second attention model using the structural information of the plurality of drugs and the gene expression information to obtain a fourth weight matrix, a fifth weight matrix and a sixth weight matrix.

18-23. (canceled)

24. A non-transitory computer-readable storage medium, the storage medium being configured to store computer program instructions, wherein when the computer program instructions are run, the method for predicting drug sensitivity according to claim 1.

25. A device for predicting drug sensitivity, comprising a first memory, a first processor, and a computer program stored on the first memory and runnable on the first processor, to perform:
acquiring gene expression information of a cell line to be tested, gene mutation information of the cell line to be tested, and structural information of a drug to be tested;
calculating first correlation information between the structural information of the drug to be tested and the gene expression information based on a first attention model; and calculating second correlation information between the structural information of the drug to be tested and the gene mutation information based on a second attention model;
splicing the first correlation information and the second correlation information to obtain a splicing result; and
performing a prediction processing on the splicing result based on a drug sensitivity prediction model to obtain sensitivity information of the cell line to be tested for the drug to be tested.

26. (canceled)
